(19) United States
(12) Patent Application Publication
(10) Pub. No.: US 2026/0021265 A1
(43) Pub. Date: Jan. 22, 2026

(54) VENTILATION APPARATUS WITH A PROPORTIONAL SOLENOID VALVE HAVING CONTROLLED OPENING

(71) Applicant: Air Liquide Medical Systems, Antony (FR)

(72) Inventor: Taraq BECHIKHI, Livry sur Seine (FR)

(21) Appl. No.: 19/262,479

(22) Filed: Jul. 8, 2025

(30) Foreign Application Priority Data
Jul. 19, 2024 (FR) FRFR2407952

(51) Int. Cl. A61M 16/00; A61M 16/08; A61M 16/20

(52) U.S. Cl. CPC A61M 16/024; A61M 16/0875; A61M 16/203

(57) ABSTRACT

The disclosure concerns a ventilation apparatus (1) having an internal gas circuit (2) in order to convey a gas, and at least one proportional solenoid valve (4, 40, 400) with an orifice (4.1) for passage of gas with an adjustable degree of opening, arranged on the internal gas circuit (2), and comprising a coil (4.2) which is supplied electrically. An electronic control (5) controls the degree of opening of the orifice for passage of gas (4.1) of the proportional solenoid valve (4, 40, 400), by acting on the electrical current supplied to the coil (4.2) of the proportional solenoid valve (4, 40), to supply a desired flow or pressure of gas. The electronic control (5) comprise an 10 electronic circuit (10) which is configured to adjust the intensity of the electrical current supplied to the coil (4.2) according to a predetermined voltage set point (Vset).

### CROSS REFERENCE TO RELATED APPLICATIONS

This application claims the benefit of priority under 35 U.S.C. § 119 (a) and (b) to French Patent Application No. FR2407952, filed Jul. 19, 2024, the entire contents of which are incorporated herein by reference.

### BACKGROUND

The invention concerns a ventilation apparatus comprising one or a plurality of proportional solenoid valves, the opening of which is controlled thanks to a particular electronic circuit which makes it possible to adjust the intensity of the electrical current supplied to the coil of the proportional solenoid valve(s) according to a voltage set-point.

In medical ventilation apparatuses, also known as artificial respirators or medical ventilators, use is made of one or more proportional solenoid valves in order to control, i.e., regulate or adjust the pressures and/or the flows of gas within the apparatus.

For this purpose, the degree of opening (i.e. the diameter, size or dimensions) of the orifice for passage of the gas (i.e. the calibrated orifice) of the proportional solenoid valve is varied proportionally according to the pressure or the flow required.

In order to be able to modify the degree of opening of the orifice, for example its diameter/size, the electrical current is varied in the coil of the solenoid valve, by controlling the voltage of the coil either as variable voltage, or as fixed voltage with chopping of the current.

In other words, the control of one or more proportional solenoid valves is habitually based on control, by an electronic circuit, of the electrical supply of its coil with a variable or constant voltage, and with chopping of the current of the coil with a pulse width modulated rectangular signal, such as to vary the current sent to the coil, and control the opening of the orifice of the solenoid valve.

Examples of controlled ventilators according to the prior art are given by WO00/32261, WO20214/210552 and WO20214/210566.

The disadvantage of these ways of controlling the coil of a proportional solenoid valve is the lack of compensation for the systematic drift of the current (i.e. intensity) regulated when the coil is heated by joule effect, and/or because of an increase in the ambient temperature.

However, any increase in the temperature of the coil gives rise to an increase in its resistance (R), with a correlated decrease in the current (i.e. intensity I) in the coil (i.e. Ohm's law). A consequence of this drift/decrease in the current (I) is a drift in the degree of opening of the orifice of the proportional solenoid valve, resulting in poor regulation of the pressure of the flow of gas passing through this proportional solenoid valve.

The present invention comes within this context, and its objective is to eliminate this problem in order to improve the control of the proportional solenoid valve(s) equipping a ventilation apparatus, i.e. an artificial respirator or medical ventilators, such as to supply a more precise pressure or flow of gas.

### SUMMARY

A solution according to the invention concerns a ventilation apparatus or medical ventilator comprising:
an internal gas circuit in order to convey a gas to be administered to a patient;
at least one proportional solenoid valve comprising an orifice for passage of gas with an adjustable degree of opening, the said at least one proportional solenoid valve being arranged on the said internal gas circuit, and comprising a coil which is supplied electrically; and
electronic control means which are configured to control the degree of opening of the orifice for passage of gas of the said at least one proportional solenoid valve, by acting on the electrical current supplied to the coil of the said at least one proportional solenoid valve, such as to supply a desired flow or pressure of gas.

In addition, in the ventilation apparatus, the electronic control means comprise an electronic circuit which is configured to adjust the intensity of the electrical current supplied to the coil according to a predetermined voltage set point.

Depending on the embodiment in question, the ventilation apparatus according to the invention may comprise one or more of the following characteristics:
it comprises a source of gas to be administered to a person, i.e. a patient;
the source of gas comprises a motorised turbine;
the motorised turbine is controlled by the electronic control means, in particular the acceleration and/or braking of the motor;
alternatively, the source of gas comprises gas added via an external pneumatic supply, typically a wall socket which supplies gas coming from gas piping, such as piping of a hospital network. In this case, the control means are also used to regulate the flow and/or the pressure of the gas supplied by the external pneumatic supply by action on valve means arranged on the gas circuit, typically the inspiratory branch;
the electronic control means comprise at least one electronic board bearing at least one (micro) processor which implements one or more algorithms;
it comprises electrical current supply means;
the electrical current supply means comprise an internal rechargeable battery, and/or means for connection to the mains (110/220 V), such as electrical cables, an electrical socket or the like;
it comprises a peripheral shell or casing;
the electronic control means, the solenoid valve(s) and the gas circuit are arranged in the shell of the apparatus;
the gas circuit has a single branch comprising only one inspiratory branch;
preferably, the gas circuit has a double branch, i.e. it comprises an inspiratory branch to convey the respiratory gas to the patient, and an expiratory branch to discharge the gases exhaled which are rich in CO₂;
it comprises a main proportional solenoid valve arranged on the inspiratory branch of the gas circuit;
it comprises a PEP proportional solenoid valve (i.e. PEP valve) arranged on the expiratory branch of the gas circuit;
it comprises a secondary proportional solenoid valve arranged on an oxygen intake line, making it possible to introduce supplementary oxygen which can be added to the flow of air coming from the source of gas, typically from the turbine, in order to enrich it in oxygen;
the electronic control means are configured to control the degree of opening of the orifice for passage of gas of the main proportional solenoid valve, the PEP proportional solenoid valve, and/or the secondary proportional solenoid valve;
the electronic control means are configured to control the degree of opening of the orifice for passage of gas by regulating the current in the coil of the main proportional solenoid valve, the PEP proportional solenoid valve, and/or the secondary proportional solenoid valve;
a shunt resistor is arranged in the electronic circuit, downstream from the coil such as to be able to measure;
the electronic circuit comprises a first and second resistor arranged downstream from the coil and/or the shunt resistor;
the first and second resistors are configured to combine a voltage (Vs) of the current at the terminals of the shunt resistor with the set point voltage (Vset), and to obtain a combined voltage (Vfb) which supplies a counter-reaction input of the DC/DC converter;
the gas circuit can also comprise pressure and/or flow sensors which cooperate with the control means, in order to regulate the supply of gas, in particular the control of the source of gas;
the gas circuit supplies a patient respiratory interface, such as a respiratory mask, which supplies the respiratory gas to the airways of the patient;
the gas circuit of the apparatus is connected to the patient respiratory interface by one or more flexible tubes.

### BRIEF DESCRIPTION OF THE DRAWINGS

The invention will now be better understood from the following detailed description, provided by way of non-limiting illustration, with reference to the appended figures, in which:

FIG. 1 is a process diagram of an embodiment of a respiratory assistance apparatus according to the invention;

FIG. 2 schematises an embodiment of the regulation loop which permits control of the current of the coil of the solenoid valve of a respiratory assistance apparatus according to the invention, such as the one in FIG. 1.

### DETAILED DESCRIPTION OF THE PREFERRED EMBODIMENTS

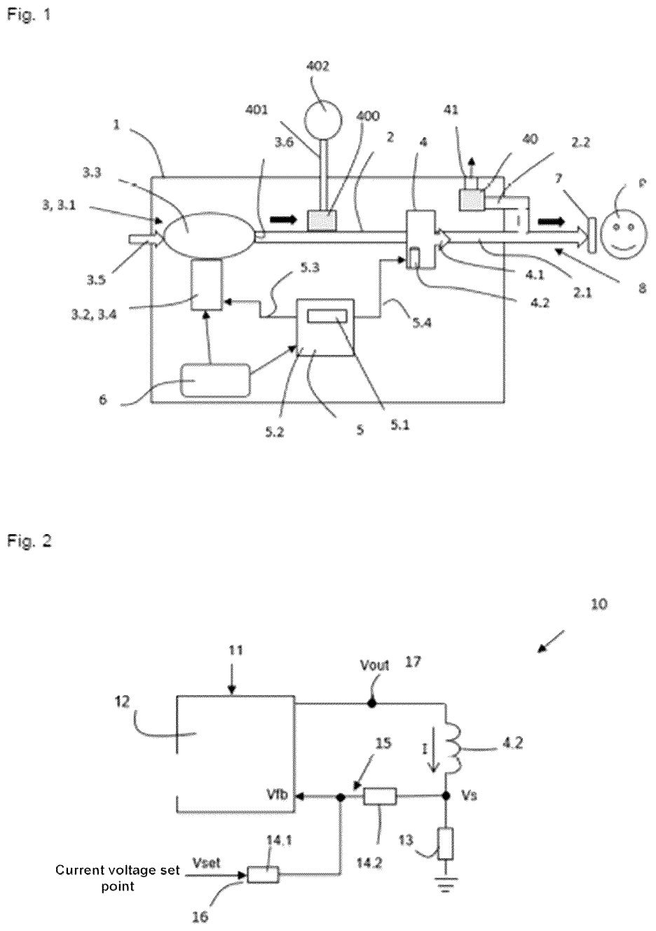

FIG. 1 schematises an embodiment of a respiratory assistance apparatus 1 according to the invention, comprising an external shell 1.1, in which there are arranged the components permitting satisfactory operation of the apparatus 1, when it is used to supply a respiratory gas (i.e. a gas with one or more components), such as air, oxygen, a mixture of air/oxygen or another gas, to a patient.

Thus, the apparatus 1 comprises an internal gas circuit 2, i.e. one or more ducts or passages for gas, in order to convey the gas to be administered to a patient P by means of a patient respiratory interface, such as a respiratory mask 7 or the like.

The gas circuit 2 of the apparatus 1 is connected to the patient respiratory interface 7 by one or more flexible tubes 8.

In this case, the internal gas circuit 2 has a double branch, i.e. it comprises an inspiratory branch 2.1 which is used to convey the gas to the patient, and an expiratory branch 2.2 which is used to recuperate the exhaled gases which are rich in CO₂. However, an internal gas circuit 2 with a single branch can also be envisaged.

The expiratory branch 2.2 makes it possible to expel the exhaled gases rich in CO₂ to the atmosphere, via an output orifice 41 which communicates fluidically with the ambient environment. The expiratory branch 2.2 also comprises a PEP valve 40, which makes it possible to adjust the expiratory pressure (PEP=positive expiratory pressure). The PEP valve 40 can be a proportional solenoid valve.

The gas, which in this case is air, is supplied to the gas circuit 2 by a source of gas 3, i.e. in this case a motorised turbine 3.1 (also known as a (micro) blower, pump, compressor or the like), i.e. equipped with an electric motor 3.2, arranged in a protective housing 3.4, which is surmounted by a volute 3.3 in which a wheel with fins (not shown) is rotated by the shaft of the motor 3.2 during its operation. The air is aspirated by the turbine 3.1, and penetrates into it via an air input 3.5 and exits from it via an output 3.6 in fluidic communication with the gas circuit 2.

However, according to another embodiment, the source of gas 3 could be a pneumatic source of gas (not shown), i.e. one or more wall sockets which supply the gas, which would be connected to gas intake piping, such as a hospital network. In this case, the internal circuits 2 of the apparatus 1 would be equipped with means for control by a controlled valve or valves, as explained hereinafter.

Optionally, the apparatus 1 can also comprise an oxygen intake line 401, making it possible to add oxygen to the air aspirated by the turbine 3.1. This addition of oxygen can be carried out upstream or downstream (cf. FIG. 1) from the turbine 3.1, according to the embodiment selected, preferably upstream from the turbine 3.1, such that a mixture of air/oxygen penetrates into the volute at 3.3 and is then conveyed to the patient via the gas circuit 2. The oxygen is obtained from an external source of oxygen 402, such as a gas cylinder or a wall socket which supplies oxygen conveyed to the wall socket by gas piping, such as a hospital network. In this case, the oxygen intake line is generally provided with a control valve, typically a secondary proportional solenoid valve 400.

In addition, as illustrated in FIG. 1, the internal gas circuit 2, in particular the inspiratory branch 2.1, comprises (at least) one main proportional solenoid valve 4 comprising an orifice for passage of gas 4.1 with an adjustable degree of opening, i.e. with a cross-section of passage of the gas which is variable and modifiable, and a coil 4.2 which is supplied electrically. The proportional solenoid valve 4 makes it possible to control the pressure and/or the flow of the gas which passes through it in the direction of the patient P (direction of the gas schematised by black arrows).

It should be noted that the other proportional solenoid valves 4, 40, 400, i.e. the one 40 which is used to control the PEP, and the one 400 which controls the additional supply of oxygen, can have the same configuration and the same operation as the main proportional solenoid valve 4 arranged on the inspiratory branch 2.1, i.e. an adjustable degree of opening, and a coil which is supplied electrically. They are also controlled by the electronic control means 5 of the apparatus 1.

In fact, the apparatus 1 also comprises electronic control means 5 comprising at least one microprocessor 5.1, such as a microcontroller, arranged on an electronic board 5.2, and implementing one or more control algorithms. The control means 5 make it possible in particular to control 5.3 the operation of the turbine 2, typically its accelerations and deceleration (i.e. braking operations) or the opening of the means for control with a controlled valve or valves, in the case of a pneumatic source of gas, as previously described.

In general, the electrical current supply means 6 supply the electrical current necessary for the satisfactory operation of the apparatus 1, in particular to the electronic control means 5. They conventionally comprise an internal rechargeable battery and/or means for connection to the mains (110/220 V), such as electrical cables, an electrical socket or the like.

It will be appreciated that the gas circuit 2 can also comprise pressure and/or flow sensors which cooperate with the control means 5, in order to regulate the supply of gas, in particular to control the source of gas 3.

A respiratory assistance apparatus architecture 1 of this type is globally conventional.

According to the invention, the electronic control means 5 also make it possible to command or control the degree of opening of the orifice for passage of gas 4.1 of some or all of the proportional solenoid valves of the apparatus 1, i.e. of the main proportional solenoid valve 4 which is arranged on the inspiratory branch 2.1, of the PEP proportional solenoid valve 40 which controls the PEP arranged on the expiratory branch 2.2, or also the secondary proportional solenoid valve 400 arranged on the optional oxygen input line 401.

By way of illustration, it is considered in the embodiment of FIG. 1 that the electronic control means 5 control the main proportional solenoid valve 4.

In this case, according to the invention, the main proportional solenoid valve 4 is controlled by acting on the electrical current supplied to the coil 4.2 of the proportional solenoid valve 4, such as to supply a desired flow or pressure of gas, including in the case of untimely heating of the coil 4 by joule effect and/or by an increase in the ambient temperature, giving rise to an increase in the resistance R, as explained hereinafter in association with FIG. 2.

For this purpose, the control means 5 comprise an electronic circuit 10 which is configured to adjust the intensity of the electrical current supplied to the coil 4.2, according to a predetermined voltage set point.

FIG. 2 describes an embodiment of an electronic circuit 10 of this type. It comprises an electrical supply input 11, downstream from which there is a Direct/Direct (DC/DC) voltage converter 12, known as the DC/DC converter. Any DC/DC converter available from semiconductor manufacturers can be used.

The current I in the coil 4.2 of the solenoid valve 4 is controlled thanks to the use of the DC/DC converter 12, which is configured to permit regulation of the current I instead of voltage regulation.

The voltage Vout at the output of the DC/DC converter 12 supplies the coil 4.2 of the solenoid valve 4, which is arranged downstream from the DC/DC converter 12.

The intensity of the current I which exits from the DC/DC converter 12, and circulates in the coil 4.2, is measured thanks to a shunt resistor 13. In other words, the shunt resistor 13 makes it possible to measure the current I passing through the coil 4.2 of the main solenoid valve 4.

The voltage Vs of the current at the terminals of the shunt resistor 13 is proportional to the current I in the coil 4.2.

The electronic circuit 10 also comprises a first 14.1 and a second 14.2 resistor, arranged downstream from the shunt resistor 13.

The voltage Vs of the current is combined with the set point voltage (Vset), via the two resistors 14.1, 14.2, and a combined voltage, known as Vfb is then obtained, which supplies a counter-reaction input 15 of the DC/DC converter 12, allowing the DC/DC converter 12 to adapt its output voltage Vout in order to comply with the current set point (i.e. desired intensity).

The voltage set point (Vset) corresponds to the control voltage of the regulation algorithm of the apparatus. It depends on the desired pressure or flow, which is set by the user. It is input (at 16) at the first resistor 14.1.

More specifically, the determination of the voltage set point Vset from the desired set point current I takes place as follows within the control means, i.e. microprocessor.

The intensity of the current I in the coil 4.2 is equal to:

$$I = V s / Rs$$

where
Vs is the voltage at the terminals of the shunt 13;
Rs is the resistance value of the shunt resistor 13.
   The following is then obtained:

$$V s = {V fb}^{*} ( R 1 + R 2 ) / R 1 - {V set}^{*} R 2 / R 1$$

where
Vfb is the reference voltage of the internal counter-reaction loop of the DC/DC converter 12;
R1 is the resistance value of the first resistor 14.1;
R2 is the resistance value of the second resistor 14.2;
Vset is the voltage set point value.

The following is thus obtained:

$$I = {V fb}^{*} ( R 1 + R 2 ) / ( R {1}^{*} Rs ) - {V set}^{*} ( R 2 / R {1}^{*} Rs )$$

where I is the required current for regulation of the desired flow or pressure, such that

$${V set}^{*} ( R 2 / R {1}^{*} Rs ) = {V fb}^{*} ( R 1 + R 2 ) / ( R {1}^{*} Rs ) - I$$

therefore Vset=(Vfb*(R1+R2)/(R1*Rs)−I)/(R2/R1*Rs)

In other words, the voltage Vset is determined from the desired current I.

The voltage Vset is then applied at the input 16 of the first resistor 14.1, as explained above.

On the basis of this voltage Vset, the control means can then control the circuit 10 in order to supply the desired current I, which is used to control the degree of opening of the orifice for passage of the gas 4.1 from the said at least one proportional solenoid valve 4.

This makes it possible to improve the control of the proportional solenoid valve(s) 4, 40, 400 which equip(s) the ventilation apparatus 1, and thus obtain the supply of a more precise pressure or flow of gas by the ventilator 1.

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

What is claimed is:

1. A ventilation apparatus (1) comprising:
an internal gas circuit (2) configured to convey a gas to be administered to a patient;
at least one proportional solenoid valve (4, 40, 400) comprising an orifice (4.1) adapted for passage of the gas with an adjustable degree of opening, the at least one proportional solenoid valve (4, 40, 400) being arranged on the internal gas circuit (2), and comprising a coil (4.2) which is supplied with an electrical current; and
an electronic control (5) which is configured to control the degree of opening of the orifice adapted for passage of the gas (4.1) of the at least one proportional solenoid valve (4, 40, 400), by acting on the electrical current supplied to the coil (4.2) of the at least one proportional solenoid valve (4, 40), to thereby supply a desired flow or pressure of gas,
characterised in that the electronic control (5) comprises an electronic circuit (10) which is configured to adjust the intensity of the electrical current supplied to the coil (4.2) according to a predetermined voltage set point (Vset).

2. The apparatus according to claim 1, characterised in that the electronic circuit (10) comprises a Direct/Direct voltage converter (12).

3. The apparatus according to claim 1, characterised in that the apparatus comprises a main proportional solenoid valve (4) arranged on an inspiratory branch (2.1) of the gas circuit (2).

4. The apparatus according to claim 1, characterised in that the apparatus comprises a PEP proportional solenoid valve (40) arranged on an expiratory branch (2.2) of the gas circuit (2).

5. The apparatus according to claim 1, characterised in that the apparatus comprises a secondary proportional solenoid valve (400) arranged on an oxygen intake line (401).

6. The apparatus according to claim 3, characterised in that the electronic control (5) is configured to control the degree of opening of the orifice (4.1) adapted for passage of the gas of the main proportional solenoid valve (4).

7. The apparatus according to claim 6, characterised in that the electronic control (5) is configured to control the degree of opening of the orifice (4.1) adapted for passage of gas by regulating the electric current in the coil (4.2) of the main proportional solenoid valve (4).

8. The apparatus according to one of claim 2, characterised in that a shunt resistor (13) is arranged in the electronic circuit (10), downstream from the coil (4.2).

9. The apparatus according to claim 8, characterised in that the electronic circuit (10) comprises a first and second resistor (14.1, 14.2) arranged downstream from the coil (4.2) and/or the shunt resistor (13).

10. The apparatus according to claim 9, characterised in that the first and second resistors (14.1, 14.2) are configured to combine a voltage (Vs) of the electric current at the terminals of the shunt resistor (13) with the set point voltage (Vset), and to obtain a combined voltage (Vfb) which supplies a counter-reaction input (15) of the DC/DC converter (12).
